(19) United States
(12) Patent Application Publication
(10) Pub. No.: US 2022/0310241 A1
(43) Pub. Date: Sep. 29, 2022

(54) CLINICAL DECISION SUPPORT SCHEDULING AND ALERTS

(71) Applicant: KONINKLIJKE PHILIPS N.V., EINDHOVEN (NL)

(72) Inventors: LOUIS NICOLAS ATALLAH, BOSTON, MA (US); MINNAN XU, CAMBRIDGE, MA (US); PAYAAL PATEL, READING, MA (US); VALERIA PANNUNZIO, EINDHOVEN (NL); CORNELIS CONRADUS ADRIANUS MARIA VAN ZON, EVERETT, MA (US)

(21) Appl. No.: 17/641,480

(22) PCT Filed: Sep. 11, 2020
(86) PCT No.: PCT/EP2020/075460

§ 371 Date: Mar. 9, 2022

Related U.S. Application Data
(60) Provisional application No. 62/900,854, filed on Sep. 16, 2019.

(51) Int. Cl. G16H 40/20; G16H 10/60; G16H 50/30

(52) U.S. Cl. CPC G16H 40/20; G16H 10/60; G16H 50/30

(57) ABSTRACT

Methods and systems for adaptive clinical decision support (CDS). The system may include a CDS score calculator configured to calculate a plurality of patient CDS scores, a detector configured to determine at least one clinician location or resource location, a scheduler configured to receive the plurality of CDS scores, receive the at least one clinician location or resource location, generate a patient priority list based on the plurality of CDS scores and the at least one clinician location or resource location, and generate, using the patient priority list, a patient schedule for at least one recipient, and a transmitter configured to transmit an alert to the at least one recipient if the generated schedule is different from a previously generated schedule.

### TECHNICAL FIELD

Embodiments described herein generally relate to systems and methods for generating a clinician schedule and, more particularly but not exclusively, to systems and methods for generating clinical decision support (CDS) schedules.

### BACKGROUND

In a hospital environment, CDS systems may be used to provide clinicians, staff, patients, or other individuals with knowledge and person-specific information, intelligently filtered or presented at appropriate times, to enhance patient health and health care.

However, CDS systems may add a new layer of alarms to the busy schedules of hospital clinicians. Electronic medical record providers have begun to provide algorithms to alert clinicians and nurses when they are charting patient data on the electronic medical record. By integrating different CDS algorithms into the electronic medical record, nurses and clinicians can be alerted when charting to a certain increased risk, such as that of sepsis or kidney injury.

Although CDS algorithms may help in healthcare, there are drawbacks to current CDS systems. Assuring compliance with CDS alerts can present a hurdle when integrating CDS in clinical workflow. Alerts may only be shown on certain clinical visualization tools, such as a specific electronic medical record for a patient. If a clinician is logged into a patient's medical record, the clinician would receive an alert. However, if a clinician does not have access to a patient's medical record, the alert for the clinician may be delayed or missed.

Additionally, reliance on manual input into a medical chart may result in an alert detection delay. Clinical events may happen between periods of charting. If an event happens between periods of charting, the patient's electronic medical record may not be properly updated until a subsequent charting period and an associated alert may also be consequently delayed.

Moreover, clinicians may not know how to act on a certain alert from a newly integrated CDS system or may otherwise be improperly alerted on an implemented CDS system. If the medical record of a patient indicates that the patient needs care, a second medical record for a second patient may also indicate the same issue. Patients in the hospital may have different needs and these simultaneous needs may cause scheduling conflicts. Patient needs are often ranked by acuity, but not every need from a patient who is recovering from a heart attack must be addressed by a pulmonary expert.

Furthermore, CDS systems may add to alarm fatigue in a hospital setting if integrated into the regular hospital alert system. Hospitals have alerts for a patient's heart rate, breathing rate, and blood pressure. After hearing many alerts, clinicians may ignore alerts or may assume other clinicians may address an alert. Ignoring or inappropriately delaying a response to these alerts or a subsequent CDS-generated alert may result in the delay of a patient's medical care.

A need exists, therefore, for methods and systems that overcome the above disadvantages of existing CDS systems integrated into clinical workflow.

### SUMMARY

This summary is provided to introduce a selection of concepts in a simplified form that are further described below in the Detailed Description section. This summary is not intended to identify or exclude key features or essential features of the claimed subject matter, nor is it intended to be used as an aid in determining the scope of the claimed subject matter.

In one aspect, embodiments relate to an adaptive clinical decision support scheduling system. The system includes a CDS score calculator configured to calculate a plurality of patient CDS scores; a detector configured to determine at least one clinician location or resource location; a scheduler configured to: receive the plurality of CDS scores; receive the at least one clinician location or resource location; generate a patient priority list based on the plurality of CDS scores and the at least one clinician location or resource location; and generate, using the patient priority list, a patient schedule for at least one recipient; and a transmitter configured to transmit an alert to the at least one recipient if the generated schedule is different from a previously generated schedule.

In some embodiments, the CDS score calculator is configured to continuously calculate the plurality of CDS scores. In some embodiments, the detector is configured to continuously detect the at least one clinician location or resource location. In some embodiments, the system includes an interface permitting the at least one recipient to supply a response. In some embodiments, the at least one clinician location or resource location is updated in substantially real time.

In some embodiments, the alert includes a severity level. In some embodiments, at least one CDS score calculator calculates the plurality of CDS scores using at least one of body temperature, pulse rate, respiration rate, blood pressure, lactate reading, serum creatinine reading, medication dose, and medical procedure of a patient. In some embodiments, the system is configured to display at least one factor leading to the alert. In some embodiments, the factor leading to the alert is a result of a detected reading below a calculated threshold. In some embodiments, the factor leading to this alert is a result of a change of the factor over a period of time. In some embodiments, the system includes an interface wherein the recipient can snooze the alert. In some embodiments, the system includes an interface configured to display a graph of CDS scores of a patient over time.

In another aspect, embodiments relate to an adaptive method for generating clinical decision support schedules. The method includes calculating, with a CDS score calculator, a plurality of patient CDS scores; determining, with a detector, at least one clinician location or resource location; receiving, with a scheduler, the plurality of CDS scores; receiving, with the scheduler, the at least one clinician location or resource location; generating, with the scheduler, a patient priority list based on the plurality of CDS scores and the at least one clinician location or resource location; generating, with the scheduler and the patient priority list, a patient schedule for at least one recipient; and transmitting, with a transmitter, an alert to the at least one recipient if the generated schedule is different from a previously generated schedule.

In some embodiments, the method includes continuously calculating the plurality of CDS scores. In some embodiments, the method includes continuously detecting the at least one clinician location or resource location. In some embodiments, the method includes receiving, with the scheduler, a response from the at least one recipient. In some embodiments, the method includes updating the at least one clinician location or resource location in substantially real time. In some embodiments, the alert includes a severity level. In some embodiments, at least one CDS score is calculated using at least one of the body temperature, pulse rate, respiration rate, and blood pressure of a patient. In some embodiments, the method includes displaying at least one factor leading to the alert. In some embodiments, the method includes displaying, with an interface, a first graph of CDS scores of a patient over time and a second graph of at least one patient factor over time

### BRIEF DESCRIPTION OF DRAWINGS

Non-limiting and non-exhaustive embodiments of the invention are described with reference to the following figures, wherein like reference numerals refer to like parts throughout the various views unless otherwise specified.

FIG. 1 illustrates a method of alerting a clinician based on the clinician location and relevant resource location in accordance with one embodiment;

FIG. 2 illustrates an alert system to facilitate task management for staff in accordance with one embodiment; and

FIG. 3 illustrates a visualization of a CDS alert and CDS indices evolving over time in accordance with one embodiment.

### DETAILED DESCRIPTION

Various embodiments are described more fully below with reference to the accompanying drawings, which form a part hereof, and which show specific exemplary embodiments. However, the concepts of the present disclosure may be implemented in many different forms and should not be construed as limited to the embodiments set forth herein; rather, these embodiments are provided as part of a thorough and complete disclosure, to fully convey the scope of the concepts, techniques and implementations of the present disclosure to those skilled in the art. Embodiments may be practiced as methods, systems or devices. Accordingly, embodiments may take the form of a hardware implementation, an entirely software implementation or an implementation combining software and hardware aspects. The following detailed description is, therefore, not to be taken in a limiting sense.

Reference in the specification to “one embodiment” or to “an embodiment” means that a particular feature, structure, or characteristic described in connection with the embodiments is included in at least one example implementation or technique in accordance with the present disclosure. The appearances of the phrase “in one embodiment” in various places in the specification are not necessarily all referring to the same embodiment. The appearances of the phrase “in some embodiments” in various places in the specification are not necessarily all referring to the same embodiments.

Some portions of the description that follow are presented in terms of symbolic representations of operations on non-transient signals stored within a computer memory. These descriptions and representations are used by those skilled in the data processing arts to most effectively convey the substance of their work to others skilled in the art. Such operations typically require physical manipulations of physical quantities. Usually, though not necessarily, these quantities take the form of electrical, magnetic or optical signals capable of being stored, transferred, combined, compared and otherwise manipulated. It is convenient at times, principally for reasons of common usage, to refer to these signals as bits, values, elements, symbols, characters, terms, numbers, or the like. Furthermore, it is also convenient at times, to refer to certain arrangements of steps requiring physical manipulations of physical quantities as modules or code devices, without loss of generality.

However, all of these and similar terms are to be associated with the appropriate physical quantities and are merely convenient labels applied to these quantities. Unless specifically stated otherwise as apparent from the following discussion, it is appreciated that throughout the description, discussions utilizing terms such as “processing” or “computing” or “calculating” or “determining” or “displaying” or the like, refer to the action and processes of a computer system, or similar electronic computing device, that manipulates and transforms data represented as physical (electronic) quantities within the computer system memories or registers or other such information storage, transmission or display devices. Portions of the present disclosure include processes and instructions that may be embodied in software, firmware or hardware, and when embodied in software, may be downloaded to reside on and be operated from different platforms used by a variety of operating systems.

The present disclosure also relates to an apparatus for performing the operations herein. This apparatus may be specially constructed for the required purposes, or it may comprise a general-purpose computer selectively activated or reconfigured by a computer program stored in the computer. Such a computer program may be stored in a computer readable storage medium, such as, but is not limited to, any type of disk including floppy disks, optical disks, CD-ROMs, magnetic-optical disks, read-only memories (ROMs), random access memories (RAMs), EPROMs, EEPROMs, magnetic or optical cards, application specific integrated circuits (ASICs), or any type of media suitable for storing electronic instructions, and each may be coupled to a computer system bus. Furthermore, the computers referred to in the specification may include a single processor or may be architectures employing multiple processor designs for increased computing capability.

The processes and displays presented herein are not inherently related to any particular computer or other apparatus. Various general-purpose systems may also be used with programs in accordance with the teachings herein, or it may prove convenient to construct more specialized apparatus to perform one or more method steps. The structure for a variety of these systems is discussed in the description below. In addition, any particular programming language that is sufficient for achieving the techniques and implementations of the present disclosure may be used. A variety of programming languages may be used to implement the present disclosure as discussed herein.

In addition, the language used in the specification has been principally selected for readability and instructional purposes and may not have been selected to delineate or circumscribe the disclosed subject matter. Accordingly, the present disclosure is intended to be illustrative, and not limiting, of the scope of the concepts discussed herein.

As mentioned previously, clinicians such as doctors, physicians, nurses, or other types of medical personnel may be alerted to a patient having an increased risk of medical complication. Commercially available CDS systems and their corresponding algorithms may pose a number of issues in a hospital setting. Available CDS systems may not be able to properly assign clinicians and resources during times of scheduling constraints. Patients have different needs and, based on the acuity of the need and past intervention success, these needs may change in urgency over the time of a patient's stay in a medical facility. Although a patient may have recently had a heart attack, not every need of that patient must be met by a pulmonary specialist. Some needs could be met by a nurse, resident, or hospital volunteer. As explained in further detail below, embodiments may account for the needs of a patient, the resources needed to treat the patient, and the people qualified to address the needs of the patient when outputting an alert.

Additionally, although a patient is in the ICU, that patient may not need attention as frequently as another patient. To improve workflow in a healthcare setting, embodiments of the CDS scheduler may manage the set of patients in a ward in order of priority, acuity, and proximity to a clinician and the necessary resources. Moreover, to combat alarm fatigue, embodiments may link an alert from a patient to a specific action needed to be taken by a clinician. For example, if a patient's heart rate is slowly declining, the system may alert a particular nurse to check the patient's heart rate, temperature, and respiratory rate within the next fifteen minutes. Some embodiments may have manual updates such that the programmed scheduled response to an alert may follow hospital or company protocol.

In some embodiments, the systems and methods described herein may deliver mobile alerts regarding an update of the status of a patient while respecting a clinician's workflow, the priority of a task, and proximity of people, caregivers and resources.

Some embodiments may schedule CDS alerts based on patient severity, as judged by CDS, clinician role, clinician locations, and available resources. Some embodiments may have a scheduler that is adaptable over time. For example, the scheduler may adapt to the pace an individual clinician can travel from one patient to the next, the average time to acquire a new resource, and the knowledge a clinician may gain over time.

Some embodiments may use a display to show a user the parameters leading to a CDS alert or alarm. Relevant parameters may be displayed based on the alert chosen by the system. In some embodiments, the parameters may be displayed to assist a clinician to understand why an alert was shown.

In some embodiments, when an alert is displayed, a clinician may be able to silence the alert. A clinician may be able to snooze the alert or otherwise delay the alert for a set period of time. In some embodiments, the clinician may be able to set the amount of time the alert may be delayed. The system may send an alert to a second clinician if the first clinician delays the alert. In some embodiments, a clinician may be able to manually input unavailability when the clinician receives an alert. If a clinician is unavailable to respond to an alert, the system may send the alert to a second clinician.

In some embodiments, the CDS parameters may be displayed over time to a clinician. The CDS parameters may be displayed in context of medical procedures or medication given to a patient over time to help the clinician understand the context of the alert. In some embodiments, the CDS parameters may be shown alongside the improvement or deterioration of a patient. A user may be able to prompt a display screen to show at least one CDS parameter graphed over time alongside at least one patient trait. For example, a user may be able to show the blood pressure of a patient over time alongside the calculated CDS score of the patient.

In some embodiments, the system may display an action to be taken for a patient when delivering an alert. For example, the system may prompt a clinician to perform a lab test or acquire an image of a patient when issuing an alert for a patient.

Some embodiments may address the issue of alarm fatigue in a hospital setting. Alarm fatigue sets in when alarms are present in a hospital such that, eventually, human responders may tune out an alarm or not appropriately respond to an alarm. Not responding to alarms at the appropriate time may result in patient needs going unmet. Alarms may be used in intensive care units, emergency settings, and other general hospital wards.

In some embodiments, CDS alerts add to the normal set of alarms received by clinicians in a hospital setting during a shift. In some embodiments, the normal set of alarms may include a breathing rate, heart rate, and the blood pressure of a patient. In some embodiments, the system may alleviate alarm fatigue by sending CDS alerts to a specific clinician that may include instructions to tend to a particular patient. In some embodiments, the CDS alerts to a specific clinician may be replacements for non-actionable alarms to other staff members.

For example, in some embodiments, the system may be used for a patient expressing acute respiratory distress syndrome (ARDS) in the intensive care unit (ICU). The detection of this syndrome is limited at this time due to the complexity of the disease, insufficient understanding of its development and progression, and the large amount of risk factors and modifiers. Improper or late detection of ARDS may result in multiple organ failure and, potentially, mortality for a patient. In some embodiments, a system using CDS alerts may analyze vital signs of a patient and may supply the vital signs to an algorithm for the early detection of ARDS. If the system detects that a patient may be exhibiting the signs of ARDS, the system may identify a team of clinicians, including bed nurses, intensivists, and pulmonary experts and a set of resources that may be used to treat the patient if necessary. The system, in some embodiments, may then develop a coordinated series of events to alert a proper team member at a relevant time to perform a particular task, schedule team members for future projected events, and identify and reserve materials and machines in nearby locations to be used for a specific patient.

Some embodiments may implement algorithms as detailed in N. W. Chbat et al., “Clinical knowledge-based inference model for early detection of acute lung injury,” Ann. Biomed. Eng., vol. 40, no. 5, pp. 1131-1141, May 2012 and A. Ahmed et al., “Development and validation of electronic surveillance tool for acute kidney injury: A retrospective analysis,” J. Crit. Care, vol. 30, no. 5, pp. 988-993, October 2015. The content of these references is hereby incorporated by reference as if set forth in their entirety herein.

In some commercially available systems, alerts may be displayed on one central machine for the medical team, a series of actions may be projected to the entire team when a team member may not be necessary for every action, and materials may not be properly identified or reserved.

By contrast, in some embodiments, systems display alarms on different machines for different team members. For example, in some commercially available systems, a patient's electronic medical record may be the only place to display an alert. If a clinician was with a different patient, the clinician may not be adequately notified that they were needed to assist with a deteriorating patient. Additionally, uploading a modified alert to a patient's medical record may take time and may delay the alert system further.

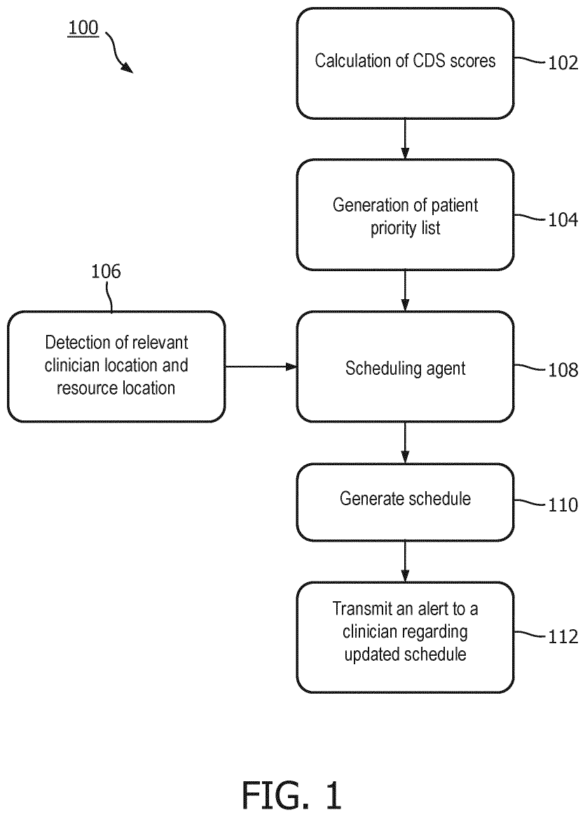

Some embodiments may alert a relevant clinician about a patient based on the severity of the patient's condition, the location of the clinical team, and resources to deliver in response to an alert as illustrated by FIG. 1. In some embodiments, the alert system may be an adaptive alert system.

In some embodiments, a CDS score calculator may be used to calculate a CDS score of each patient in a medical facility 102. In some embodiments, the CDS score calculator may be continuously updated 102. In some embodiments, the CDS score calculator 102 may receive continuous updates of vital signs from a patient and may also receive manual input from clinicians attending to the patient. In some embodiments, the CDS score calculator 102 may generate a score upon prompting by a clinician.

A CDS score may be based on at least one of the patient's vital signs, including temperature, pulse rate, respiration rate, blood pressure, lactate reading, or creatinine reading. The CDS score may be based on at least one of the patient's medication dose or a medical procedure performed on a patient. The CDS score may also include if the patient is sleeping or awake. In some embodiments, the CDS score may include the presence of visitors in the room of the patient. In some embodiments, the CDS score may be based on a plurality of the above.

In some embodiments, the system 100 may use the calculation of CDS scores 102 to generate a patient priority list 104. The patient priority list 104 may rank the needs of each patient according to the CDS score from most important to least important. The patient priority list 104 may be continuously updated with the continuous calculation of CDS scores 102.

In some embodiments, the system 100 may use a detector to detect the location of a relevant clinician and relevant resources 106 for a patient. In some embodiments, the detector may continuously update the locations of the clinicians and the resources in a ward or hospital 106. In some embodiments, a plurality of detectors may be used to determine the location of clinicians and resources in a medical facility 106. In some embodiments, detectors may only be used to determine the location of resources 106. Detectors may only be used to determine the location of clinicians. In some embodiments, the detectors may use GPS or other location-based data signaling to determine the location of a clinician or resource 106. The detector may be a phone, tablet, or pager in some embodiments. In some embodiments, the clinician location 106 may be updated in real time. The resource location 106 may be updated in real time.

In some embodiments, a scheduling agent 108 may receive the location data 106 and the patient priority list 104 to generate a schedule for at least one clinician 110. The scheduling agent may use machine learning methods, manually-inputted clinician determinations, or a combination thereof to generate the schedule 110. Overall, the scheduling agent 108 may use patient severity, staff location, and resource location to generate a medical facility schedule and a schedule for each staff member and resource within that medical facility 110. In some embodiments, the schedule 110 may be compared to a previous schedule for the clinician. If the generated schedule 110 differs from a previous schedule, an alert 112 may be transmitted to a clinician. In some embodiments, the scheduling agent 108 may calculate the schedule 110 in the cloud and transmit an alert 112 to a clinician on a mobile device, patient monitor, central station, or ICU software system.

In some embodiments, the alerts 112 may be updated continuously based on received input about the clinician's location, the workload of a clinician, the availability of a resource, the location of a resource, the status of a patient, or any combination thereof.

In some embodiments, the scheduling agent 108 may be a dynamic scheduling agent and may use optimization methods to streamline the workflow in a hospital. Some embodiments may use scheduling optimizers as detailed in J. Balasubramanian and I. E. Grossmann, “Scheduling optimization under uncertainty—an alternative approach,” Comput. Chem. Eng., vol. 27, no. 4, pp. 469-490, April 2003 and P. Skobelev, “Multi-Agent Systems for Real Time Resource Allocation, Scheduling, Optimization and Controlling: Industrial Applications,” in Holonic and Multi-Agent Systems for Manufacturing, 2011, pp. 1-14. The content of these references is hereby incorporated by reference as if set forth in their entirety herein.

In some embodiments, an alert may be transmitted to one clinician at one device 112. For example, if a clinician is located far from a patient, the system may send an alert to a pager or smartphone of a clinician to indicate that the clinician is needed. If a clinician is with a patient at the time of the alert, the alert may be sent to the patient monitor. If there is no clinician within a predetermined area to assist a patient, the system may send an alert to a central station to be addressed by a relevant clinician.

In some embodiments, if the closest clinician is not available, an alert is delayed, or a resource is not available, the system may reconfigure the schedule to make another clinician available or to make a resource available if the severity of the need necessitates the reconfiguration. In some embodiments, a clinician may receive an updated schedule 110 or an updated alert 112 for a patient when the acuity of a patient's need has changed.

In some embodiments, the alert 112 may include the severity level of a patient. In some embodiments, the severity level may include the amount of time a clinician may have to respond. The alert 112 may be displayed with additional warnings if the patient's need has increased.

For example, if a patient may be suffering from ARDS, a system may issue an alert first to a bed nurse and intensivist. Depending on the severity of the patient's need, the success of the original clinicians, and the availability of the bed nurse and intensivist, the system may then send an alert to a pulmonologist.

In some embodiments, the system may determine that a patient may be showing early signs of sepsis. If the patient is exhibiting these signs, the system may only alert a bed nurse and not an intensivist or pulmonologist. The bed nurse may then respond to the alert and input observations into the system.

In another example, if a patient is exhibiting signs of a deep vein thrombosis, an alert may be sent to both a bed nurse and a central nurse. The bed nurse and the central nurse may both respond to the alert or only one may need to respond, based on the protocol of the hospital.

In some embodiments, the type of alert may be pre-programmed with the ideal type of clinician to respond to the alert and the ideal materials needed to respond to the alert. All personnel in the hospital may be assigned into a clinician category and then the system may assign a person from a clinician category to respond to an alert. In some embodiments, some examples of clinician categories may be hospital volunteer, bed nurse, central nurse, registered nurse, medical student, attending, resident physician, and specialist physician.

For example, if the patient only needed to be looked over for signs of sepsis, the system may note that a clinician with any amount of experience could be delegated to the task. This could be a bed nurse, a central nurse, a resident, or a specializing physician. The rank of clinician priority could correspond to the specialization rank, such that a bed nurse would get assigned to sepsis checks more often than an attending physician because the attending physician may be needed to respond to other specialized matters. In some embodiments, if a patient needed to be looked over because of a dropping breathing rate, an attending physician or specialist doctor may be the only two types of clinician categories designated as qualified to respond.

In some embodiments, the scheduling agent 108 may send an alert based on location, the severity of the patient, or the resources required to properly respond to the patient 110. In some embodiments, the location may mean the closest bed nurse or the closest clinician capable of responding to the alert. In some embodiments, the severity of the patient and the patient priority list 104 may include alarms such as a dropping heart rate, early signs of potential sepsis, and potential deep vein thrombosis.

In some embodiments, the system may be adapted for specific hospital wards. For example, some clinicians in a certain ward may agree to certain thresholds for different CDS algorithms. In some embodiments, the thresholds may be determined automatically by a statistical/machine learning method that identifies thresholds that have led to adverse events in previous data. Adverse events may mean patient deterioration or death.

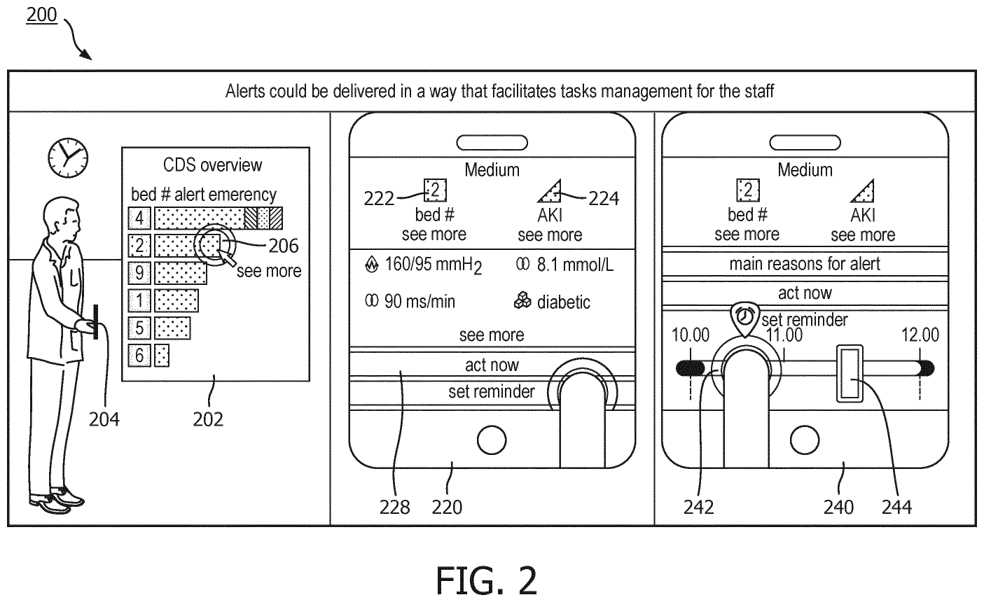

As shown in FIG. 2, an alert may be delivered in a way that facilities task management for staff 200. In some embodiments, a clinician may receive a CDS overview 202 for a patient on a device 204. In some embodiments, the device may be a tablet, pager, cell phone, or patient monitor. The alert may be associated with a patient, the patient being assigned a specific bed number. In some embodiments, the acuity of the alert may be shown based on bedside urgency. In some embodiments, the clinician may be able to interact with the alert 206. The alert 206 may display at least one factor leading to the alert on the first screen 202 or second screen 200, providing the main reasons for the alert 226.

In some embodiments, the clinician may interact with the alert 206 and may request more information. The clinician may want to see other reasons for the alert, the main reasons for the alert 226, the medical history of the patient, and/or the time the clinician has to respond to the alert 228. In some embodiments, all of this information may be displayed on a single screen 220. In some embodiments, the information may be available on different screens or in drop-down menus. In some embodiments, a user may be able to click or swipe with their finger or a stylus for additional information. In some embodiments, the request for more information may lead the clinician to a secondary screen 220. The secondary screen may display the sense of urgency 224, the location of the patient 222, the reasons for the alert 226, and an alert scheduler 228. In some embodiments, some of the reasons for the alert and the severity for the alert may be color-coded. For example, if the patient's preexisting condition of diabetes may be contributing to an alert urgency, the pre-existing condition may appear to be green because the condition had not changed since the patient arrived at the hospital. However, a high blood pressure may appear in red because the blood pressure had gone up and was outside of the normal vital level of the patient.

In some embodiments, the secondary screen showing alerts per CDS index can be customized to be specific to the condition. For example, a hemodynamic instability alert may show a patient's blood pressure and heart rate among other parameters whereas an acute kidney injury may show updated lab results and the likely reason for the kidney injury.

In some embodiments, the clinician may be able to interact with the alert by selecting “act now” or “set reminder” 228 to address the alert at a later time. If the clinician chooses to address the alert at a later time, the clinician may be directed to a third screen 240 in some embodiments or a dropdown window.

In some embodiments, the clinician may be able to delay the alarm by a certain amount of time. The display screen may show a set amount of time where the alert could be delayed without harm to the patient. In some embodiments, the section of a non-harmful time delay may be highlighted in green 242. In some embodiments, the longer the response delay, the greater potential for harm to the patient. In some embodiments, if a clinician wants to delay the alert past a certain time period, the alert system may indicate that the delay may present a harmful delay 244. In some embodiments, the harmful time delay 244 may be highlighted in red. In some embodiments, the snooze element 240 may help clinicians manage their workload. In some embodiments, the snooze element 240 may include a silence element to silence the alert.

In some embodiments, this set amount of time may be preset depending on the type of alert issued for a patient. For example, if a patient is exhibiting early onset of sepsis, the time window to respond may be greater than if a patient was exhibiting early signs of a potential stroke. In some embodiments, if a patient's severity level increased, the physician may receive an alert update before the reminder was set.

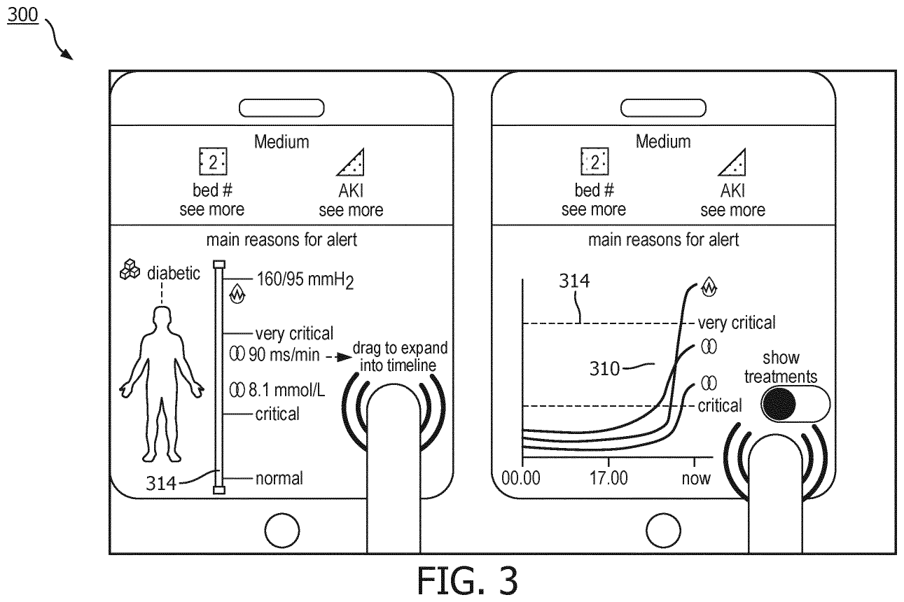

FIG. 3 illustrates a visualization of a CDS alert 300 and CDS indices evolving over time in accordance with one embodiment. In some embodiments, a clinician can determine how CDS indices evolved over time for a patient by viewing a graphical timeline of a patient's vital signs 310. The clinician may look at certain thresholds 304 to determine the critical status of a patient over time, see alerts issued for the patient, and see what treatments were done for the patient. After administration of the treatments, the clinician can see if the patient improved, maintained their status, or declined.

In some embodiments, a graph 310 may show limits 314 concerning how critical the patient is at each point of time. The clinician can then see the treatments that were done and how the index was affected to determine potential routes of future treatment and stability of the patient. In some embodiments, the graph 310 may display the CDS score of a patient over time. The graph 310 may show at least one patient factor, such as the heart rate of a patient over time. In some embodiments, the graph may show the CDS score of a patient over time alongside the at least one patient factor in a simultaneous graph 310. A user may input a request to display a certain vital sign or patient factor over time alongside a CDS score of a patient in some embodiments.

In some embodiments, a user may display the reason for an alert generated for a patient. In some embodiments, the system may generate an alert because a machine or person detected a reading of a vital sign below a calculated threshold. For example, the breathing rate for a patient may be below a certain number of repetitions per minute and the rate may trigger an alert. In some embodiments, the system may generate an alert because a machine or person detected a reading of a vital sign above a calculated threshold. For example, the heart rate for a patient may be above a certain number of repetitions per minute and the rate may trigger an alert.

In some embodiments, the system may generate an alert for a patient because a vital sign of a patient has changed over time. For example, a patient may have come in as an elite athlete with a resting heart rate of 40 beats per minute. Although this may be below average for most people, this may be recorded as a normal baseline for the patient in the system. Over a few days, the resting heart rate may increase to 80 beats per minute. Although this may still be a normal resting heart rate for the average adult, because the heart rate of the patient doubled over time, the system may issue an alert in some embodiments. A user may display the progression over time, the calculation of the CDS score, and the correlation between the vital sign and the alert in some embodiments.

The methods, systems, and devices discussed above are examples. Various configurations may omit, substitute, or add various procedures or components as appropriate. For instance, in alternative configurations, the methods may be performed in an order different from that described, and that various steps may be added, omitted, or combined. Also, features described with respect to certain configurations may be combined in various other configurations. Different aspects and elements of the configurations may be combined in a similar manner. Also, technology evolves and, thus, many of the elements are examples and do not limit the scope of the disclosure or claims.

Embodiments of the present disclosure, for example, are described above with reference to block diagrams and/or operational illustrations of methods, systems, and computer program products according to embodiments of the present disclosure. The functions/acts noted in the blocks may occur out of the order as shown in any flowchart. For example, two blocks shown in succession may in fact be executed substantially concurrent or the blocks may sometimes be executed in the reverse order, depending upon the functionality/acts involved. Additionally, or alternatively, not all of the blocks shown in any flowchart need to be performed and/or executed. For example, if a given flowchart has five blocks containing functions/acts, it may be the case that only three of the five blocks are performed and/or executed. In this example, any of the three of the five blocks may be performed and/or executed.

A statement that a value exceeds (or is more than) a first threshold value is equivalent to a statement that the value meets or exceeds a second threshold value that is slightly greater than the first threshold value, e.g., the second threshold value being one value higher than the first threshold value in the resolution of a relevant system. A statement that a value is less than (or is within) a first threshold value is equivalent to a statement that the value is less than or equal to a second threshold value that is slightly lower than the first threshold value, e.g., the second threshold value being one value lower than the first threshold value in the resolution of the relevant system.

Specific details are given in the description to provide a thorough understanding of example configurations (including implementations). However, configurations may be practiced without these specific details. For example, well-known circuits, processes, algorithms, structures, and techniques have been shown without unnecessary detail in order to avoid obscuring the configurations. This description provides example configurations only, and does not limit the scope, applicability, or configurations of the claims. Rather, the preceding description of the configurations will provide those skilled in the art with an enabling description for implementing described techniques. Various changes may be made in the function and arrangement of elements without departing from the spirit or scope of the disclosure.

Having described several example configurations, various modifications, alternative constructions, and equivalents may be used without departing from the spirit of the disclosure. For example, the above elements may be components of a larger system, wherein other rules may take precedence over or otherwise modify the application of various implementations or techniques of the present disclosure. Also, a number of steps may be undertaken before, during, or after the above elements are considered.

Having been provided with the description and illustration of the present application, one skilled in the art may envision variations, modifications, and alternate embodiments falling within the general inventive concept discussed in this application that do not depart from the scope of the following claims.

1. An adaptive clinical decision support (CDS) scheduling system comprising:
a CDS score calculator configured to calculate a plurality of patient CDS scores;
a detector configured to determine at least one of a clinician location and resource location;
a scheduler configured to:
receive the plurality of CDS scores;
receive the at least one clinician location or resource location;
generate a patient priority list based on the plurality of CDS scores and the at least one of the clinician location and the resource location; and
generate, using the patient priority list, a patient schedule for at least one recipient;
receive one or more updates, wherein the one or more updates include an update to at least one of the clinician location, clinician workload, the resource location, resource availability, a schedule change, and a patience status;
determine whether the one or more updates exceed a severity threshold;
responsive to determining that the one or more updates does not exceed the severity threshold, send a first alert to a first set of devices;
responsive to determining that the one or more updates exceeds the severity threshold, send a second alert to a second set of devices, wherein the second set of devices includes at least a first device and a second device; and
a communication module configured to:
responsive to receiving the first alert, transmit the first alert to the at least one recipient; and
responsive to receiving the second alert, transmit the second alert to at least two recipients.

2. The system of claim 1, wherein the CDS score calculator is configured to continuously calculate the plurality of CDS scores.

3. The system of claim 1, wherein the detector is configured to continuously detect the at least one the clinician location and the resource location.

4. The system of claim 1, further comprising an interface permitting the at least one recipient to supply a response.

5. (canceled)

6. The system of claim 1, wherein at least one of the first alert and the second alert comprises a severity level.

7. The system of claim 1, wherein at least one CDS score calculator calculates the plurality of CDS scores using at least one of body temperature, pulse rate, respiration rate, blood pressure, lactate reading, serum creatinine reading, medication dose, and medical procedure of a patient.

8. The system of claim 1, wherein the system is configured to display one or more factors leading to at least one of the first alert and the second alert.

9. The system of claim 8, wherein the one or more factors are a result of a detected reading below a calculated threshold.

10. The system of claim 8, wherein the one or more factors are a result of a change of the one or more factors over a period of time.

11. (canceled)

12. The system of claim 1, further comprising an interface configured to display a graph of CDS scores of a patient over time.

13. An adaptive method for generating clinical decision support (CDS) schedules comprising:
calculating, with a CDS score calculator, a plurality of patient CDS scores;
determining, with a detector, at least one of the clinician location and the resource location;
receiving, with a scheduler, the plurality of CDS scores;
receiving, with the scheduler, the at least one of the clinician location and the resource location;
generating, with the scheduler, a patient priority list based on the plurality of CDS scores and the at least one clinician location or resource location;
generating, with the scheduler and the patient priority list, a patient schedule for at least one recipient;
receiving, with the scheduler, one or more updates, wherein the one or more updates include an update to at least one of the clinician location, clinician workload, the resource location, resource availability, a schedule change, and a patient status;
determining, with the scheduler, whether the one or more updates exceed a severity threshold;
responsive to determining that the one or more updates does not exceed the severity threshold, sending, with a communication module, a first alert to a first set of devices,
responsive to determining that the one or more updates exceeds the seventy threshold, sending, with the communication module, a second alert to a second set of devices, wherein the second set of devices includes at least a first device and a second device;
responsive to receiving the first alert transmitting, with the communication module, the first alert to the at least one recipient; and
responsive to receiving the second alert, transmitting with the communication module, the second alert to at least two recipients.

14. The method of claim 13, further comprising continuously calculating the plurality of CDS scores.

15. (canceled)

16. The method of claim 13, further comprising updating at least one of the clinician location and the resource location in substantially real time.

17. The method of claim 13, wherein at least one of the first alert and the second alert includes a severity level.

18. The method of claim 13, wherein at least one CDS score is calculated using at least one of the body temperature, pulse rate, respiration rate, and blood pressure of a patient.

19. The method of claim 13, further comprising displaying one or more factors leading to at least one of the first alert and the second alert.

20. The method of claim 13, further comprising displaying, with an interlace, a first graph of CDS scores of a patient over time and a second graph of at least one patient factor over time.

21. The system of claim 1, wherein the communication module is further configured to update the patient schedule of the at least one recipient.

22. The system of claim 1, wherein the communication module is further configured to:
receive a response to the second alert from at least one of the at least two recipients, and
wherein the scheduler is further configured to:
responsive to receiving, through the communication module, the response through the communication module, update the patient schedule.

23. The system of claim 1, wherein the first device is coupled to a first display and the second device is coupled to a second display.
